(19) United States
(12) Patent Application Publication
(10) Pub. No.: US 2002/0064536 A1
(43) Pub. Date: May 30, 2002

(54) Methods of treating animals with botulinum toxin pharmaceutical compositions

(75) Inventor: Terrence J. Hunt, Anaheim Hills, CA (US)

Correspondence Address: Frank J. Uxa, Stout, Uxa, Buyan & Mullins, LLP, Suite 300, 4 Venture, Irvine, CA, 92618, US

(73) Assignee: Allergan Sales, Inc., Irvine, CA (US)

(21) Appl. No.: 10/047,058

(22) Filed: Jan. 14, 2002

Related U.S. Application Data
(63) Continuation-in-part of application No. 09/500,147, filed on Feb. 8, 2000.

(51) Int. Cl. A61K 39/08; A61K 31/715

(52) U.S. Cl. 424/247.1; 514/54

(57) ABSTRACT

Methods for treating mammals with botulinum toxin comprise administering at least one type of botulinum toxin to the mammal. The botulinum toxin may be administered in a composition having a polysacharide that stabilizes the botulinum toxin. The compositions administered to the mammals have reduced immunogenicity, and are preferably non-immunogenic. The methods may also be practiced with recombinant, or species-specific, serum albumins.

This application is a continuation-in-part application of U.S. patent application Ser. No. 09/500,147, filed Feb. 8, 2000, the content of which in its entirety is hereby incorporated by reference.

Various publications and/or references have been cited herein, the contents of which, in their entireties, are incorporated herein by reference.

### BACKGROUND

The present invention relates to methods of using pharmaceutical compositions. In particular, the present invention relates to methods of using Clostridial neurotoxin compositions, some of which have reduced immunogenicity in the animals being treated.

A pharmaceutical composition is a formulation containing one or more active ingredients as well as one or more excipients, carriers, stabilizers or bulking agents, which is suitable for administration to a patient to achieve a desired diagnostic result or therapeutic effect.

For storage stability and convenience of handling, a pharmaceutical composition can be formulated as a lyophilized (i.e. freeze dried) or vacuum dried powder which can be reconstituted with saline or water prior to administration to a patient. Alternately, the pharmaceutical composition can be formulated as an aqueous solution. A pharmaceutical composition can contain a proteinaceous active ingredient. Unfortunately, proteins can be very difficult to stabilize, resulting in loss of protein and/or loss of protein activity during the formulation, reconstitution (if required) and during the storage prior to use of a protein containing pharmaceutical composition. Stability problems can occur because of protein denaturation, degradation, dimerization, and/or polymerization. Various excipients, such as albumin and gelatin have been used with differing degrees of success to try and stabilize a protein active ingredient present in a pharmaceutical composition. Additionally, cryoprotectants such as alcohols have been used to reduce protein denaturation under the freezing conditions of lyophilization.

Albumin

Albumins are small, abundant plasma proteins. Human serum albumin has a molecular weight of about 69 kiloDaltons (kD) and has been used as a non-active ingredient in a pharmaceutical composition where it can serve as a bulk carrier and stabilizer of certain protein active ingredients present in a pharmaceutical composition.

The stabilization function of albumin in a pharmaceutical composition can be present both during the multi-step formulation of the pharmaceutical composition and upon the later reconstitution of the formulated pharmaceutical composition. Thus, stability can be imparted by albumin to a proteinaceous active ingredient in a pharmaceutical composition by, for example, (1) reducing adhesion (commonly referred to as “stickiness”) of the protein active ingredient to surfaces, such as the surfaces of laboratory glassware, vessels, to the vial in which the pharmaceutical composition is reconstituted and to the inside surface of a syringe used to inject the pharmaceutical composition. Adhesion of a protein active ingredient to surfaces can lead to loss of active ingredient and to denaturation of the remaining retained protein active ingredient, both of which reduce the total activity of the active ingredient present in the pharmaceutical composition, and; (2) reducing denaturation of the active ingredient which can occur upon preparation of a low dilution solution of the active ingredient.

As well as being able to stabilize a protein active ingredient in a pharmaceutical composition, human albumin also has the advantage of generally negligible immunogenicity when injected into a human patient. A compound with an appreciable immunogenicity can cause the production of antibodies against it which can lead to an anaphylactic reaction and/or to the development of drug resistance, with the disease or disorder to be treated thereby becoming potentially refractory to the pharmaceutical composition which has an immunogenic component.

Unfortunately, despite its known stabilizing effect, significant drawbacks exist to the use of albumin in a pharmaceutical composition. For example albumins are expensive and increasingly difficult to obtain. Furthermore, blood products such as albumin, when administered to a patient can subject the patient to a potential risk of receiving blood borne pathogens or infectious agents. Thus, it is known that the possibility exists that the presence of albumin in a pharmaceutical composition can result in inadvertent incorporation of infectious elements into the pharmaceutical composition. For example, it has been reported that use of albumin may transmit prions into a pharmaceutical composition. A prion is a proteinaceous infectious particle which is hypothesized to arise as an abnormal conformational isoform from the same nucleic acid sequence which makes the normal protein. It has been further hypothesized that infectivity resides in a “recruitment reaction” of the normal isoform protein to the prion protein isoform at a post translational level. Apparently the normal endogenous cellular protein is induced to misfold into a pathogenic prion conformation. Significantly, several lots of human serum albumin have been withdrawn from distribution upon a determination that a blood donor to a pool from which the albumin was prepared was diagnosed with Creutzfeldt-Jacob disease.

Creutzfeldt-Jacob disease (sometimes characterized as Alzheimer's disease on fast forward) is a rare neurodegenerative disorder of human transmissible spongiform encephalopathy where the transmissible agent is apparently an abnormal isoform of a prion protein. An individual with Creutzfeldt-Jacob disease can deteriorate from apparent perfect health to akinetic mutism within six months. Possible iatrogenic transmission of Creutzfeldt-Jacob disease by albumin transfusion has been reported and it has been speculated that sufficient protection against Creutzfeldt-Jacob disease transmission is not provided by the usual methods of albumin preparation which methods include disposal of blood cellular elements and heating to 60 degrees C. for 10 hours. Thus, a potential risk may exist of acquiring a prion mediated disease, such as Creutzfeldt-Jacob disease, from the administration of a pharmaceutical composition which contains human plasma protein concentrates, such as serum albumin.

Gelatin has been used in some protein active ingredient pharmaceutical compositions as an albumin substitute. Notably, gelatin is a animal derived protein and therefore carries the same risk of potential infectivity which may be possessed by albumin. Hence, it is desirable to find a substitute for albumin which is not a blood fraction, and preferably, the albumin substitute is not gelatin and is not derived from any animal source.

Botulinum Toxin

The anaerobic, gram positive bacterium Clostridium botulinum produces a potent polypeptide neurotoxin, botulinum toxin, which causes a neuroparalytic illness in humans and animals referred to as botulism. Clostridium botulinum and its spores are commonly found in soil and the bacterium can grow in improperly sterilized and sealed food containers of home based canneries, which are the cause of many of the cases of botulism. The effects of botulism typically appear 18 to 36 hours after eating the foodstuffs infected with a Clostridium botulinum culture or spores. The botulinum toxin can apparently pass unattenuated through the lining of the gut and attack peripheral motor neurons. Symptoms of botulinum toxin intoxication can progress from difficulty walking, swallowing, and speaking to paralysis of the respiratory muscles and death.

Botulinum toxin type A is the most lethal natural biological agent known to man. About 50 picograms of botulinum toxin (purified neurotoxin complex) type A is a LD₅₀ in mice. Interestingly, on a molar basis, botulinum toxin type A is 1.8 billion times more lethal than diphtheria, 600 million times more lethal than sodium cyanide, 30 million times more lethal than cobrotoxin and 12 million times more lethal than cholera. Singh, Critical Aspects of Bacterial Protein Toxins, pages 63-84 (chapter 4) of Natural Toxins II, edited by B. R. Singh et al., Plenum Press, N.Y. (1976) (where the stated LD₅₀ of botulinum toxin type A of 0.3 ng equals 1 U is corrected for the fact that about 0.05 ng of BOTOX® equals 1 unit). One unit (U) of botulinum toxin is defined as the LD₅₀ upon intraperitoneal injection into female Swiss Webster mice weighing 18-20 grams each. In other words, one unit of botulinum toxin is the amount of botulinum toxin that kills 50% of a group of female Swiss Webster mice. Seven immunologically distinct botulinum neurotoxins have been characterized, these being respectively botulinum neurotoxin serotypes A, B, C₁, D, E, F, and G, each of which is distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg which is about 12 times the primate LD₅₀ for botulinum toxin type A. The botulinum toxins apparently bind with high affinity to cholinergic motor neurons, are translocated into the neuron and block the presynaptic release of acetylcholine.

Botulinum toxins have been used in clinical settings for the treatment of neuromuscular disorders characterized by hyperactive skeletal muscles. Botulinum toxin type A was approved by the U.S. Food and Drug Administration in 1989 for the treatment of essential blepharospasm, strabismus and hemifacial spasm in patients over the age of twelve. Clinical effects of peripheral intramuscular botulinum toxin type A are usually seen within one week of injection. The typical duration of symptomatic relief (i.e. flaccid muscle paralysis) from a single intramuscular injection of botulinum toxin type A can be about three months.

Although all the botulinum toxins serotypes apparently inhibit release of the neurotransmitter acetylcholine at the neuromuscular junction, they do so by affecting different neurosecretory proteins and/or cleaving these proteins at different sites. Botulinum toxin A is a zinc endopeptidase which can specifically hydrolyze a peptide linkage of the intracellular, vesicle associated protein SNAP-25. Botulinum type E also cleaves the 25 kiloDalton (kD) synaptosomal associated protein (SNAP-25), but targets different amino acid sequences within this protein, as compared to botulinum toxin type A. Botulinum toxin types B, D, F and G act on vesicle-associated protein (VAMP, also called synaptobrevin), with each serotype cleaving the protein at a different site. Finally, botulinum toxin type C₁, has been shown to cleave both syntaxin and SNAP-25. These differences in mechanism of action may affect the relative potency and/or duration of action of the various botulinum toxin serotypes.

Regardless of serotype, the molecular mechanism of toxin intoxication appears to be similar and to involve at least three steps or stages. In the first step of the process, the toxin binds to the presynaptic membrane of the target neuron through a specific interaction between the heavy chain (H chain) and a cell surface receptor; the receptor is thought to be different for each serotype of botulinum toxin and for tetanus toxin. The carboxyl end segment of the H chain, H_{C}, appears to be important for targeting of the toxin to the cell surface.

In the second step, the toxin crosses the plasma membrane of the poisoned cell. The toxin is first engulfed by the cell through receptor-mediated endocytosis, and an endosome containing the toxin is formed. The toxin then escapes the endosome into the cytoplasm of the cell. This last step is thought to be mediated by the amino end segment of the H chain, H_{N}, which triggers a conformational change of the toxin in response to a pH of about 5.5 or lower. Endosomes are known to possess a proton pump which decreases intra endosomal pH. The conformational shift exposes hydrophobic residues in the toxin, which permits the toxin to embed itself in the endosomal membrane. The toxin then translocates through the endosomal membrane into the cytosol.

The last step of the mechanism of botulinum toxin activity appears to involve reduction of the disulfide bond joining the H and L chain. The entire toxic activity of botulinum and tetanus toxins is contained in the L chain of the holotoxin; the L chain is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane. Tetanus neurotoxin, botulinum toxin B, D, F, and G cause degradation of synaptobrevin (also called vesicle-associated membrane protein (VAMP)), a synaptosomal membrane protein. Most of the VAMP present at the cytosolic surface of the synaptic vesicle is removed as a result of any one of these cleavage events. Each toxin specifically cleaves a different bond.

The molecular weight of the botulinum toxin protein molecule, for all seven of the known botulinum toxin serotypes, is about 150 kD. Interestingly, the botulinum toxins are released by Clostridial bacterium as complexes comprising the 150 kD botulinum toxin protein molecule along with associated non-toxin proteins. Thus, the botulinum toxin type A complex can be produced by Clostridial bacterium as 900 kD, 500 kD and 300 kD forms. Botulinum toxin types B and C₁ are apparently produced as only a 500 kD complex. Botulinum toxin type D is produced as both 300 kD and 500 kD complexes. Finally, botulinum toxin types E and F are produced as only approximately 300 kD complexes. The complexes (i.e. molecular weight greater than about 150 kD) are believed to contain a non-toxin hemaglutinin protein and a non-toxin and non-toxic nonhemaglutinin protein. These two non-toxin proteins (which along with the botulinum toxin molecule can comprise the relevant neurotoxin complex) may act to provide stability against denaturation to the botulinum toxin molecule and protection against digestive acids when toxin is ingested. Additionally, it is possible that the larger (greater than about 150 kD molecular weight) botulinum toxin complexes may result in a slower rate of diffusion of the botulinum toxin away from a site of intramuscular injection of a botulinum toxin complex. The toxin complexes can be dissociated into toxin protein and hemagglutinin proteins by treating the complex with red blood cells at pH 7.3. The toxin protein has a marked instability upon removal of the hemagglutinin protein.

All the botulinum toxin serotypes are made by Clostridium botulinum bacteria as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C₁, D, and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for the known significantly lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy. Additionally, it is known that botulinum toxin type B has, upon intramuscular injection, a shorter duration of activity and is also less potent than botulinum toxin type A at the same dose level.

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine, CGRP and glutamate.

High quality crystalline botulinum toxin type A can be produced from the Hall A strain of Clostridium botulinum with characteristics of ≧3×10⁷ U/mg, an A₂₆₀/A₂₇₈ of less than 0.60 and a distinct pattern of banding on gel electrophoresis. The known Shantz process can be used to obtain crystalline botulinum toxin type A, as set forth in Shantz, E. J., et al, Properties and use of Botulinum toxin and Other Microbial Neurotoxins in Medicine, Microbiol Rev. 56: 80-99 (1992). Generally, the botulinum toxin type A complex can be isolated and purified from an anaerobic fermentation by cultivating Clostridium botulinum type A in a suitable medium. Raw toxin can be harvested by precipitation with sulfuric acid and concentrated by ultramicrofiltration. Purification can be carried out by dissolving the acid precipitate in calcium chloride. The toxin can then be precipitated with cold ethanol. The precipitate can be dissolved in sodium phosphate buffer and centrifuged. Upon drying there can then be obtained approximately 900 kD crystalline botulinum toxin type A complex with a specific potency of 3×10⁷ LD₅₀ U/mg or greater. This known process can also be used, upon separation out of the non-toxin proteins, to obtain pure botulinum toxins, such as for example: purified botulinum toxin type A with an approximately 150 kD molecular weight with a specific potency of 1-2×10⁸ LD₅₀ U/mg or greater; purified botulinum toxin type B with an approximately 156 kD molecular weight with a specific potency of 1-2×10⁸ LD₅₀ U/mg or greater, and; purified botulinum toxin type F with an approximately 155 kD molecular weight with a specific potency of 1-2×10⁷ LD₅₀ U/mg or greater.

Already prepared and purified botulinum toxins and toxin complexes suitable for preparing pharmaceutical formulations can be obtained from List Biological Laboratories, Inc., Campbell, Calif.; the Centre for Applied Microbiology and Research, Porton Down, U.K.; Wako (Osaka, Japan), as well as from Sigma Chemicals of St Louis, Mo.

It has been reported that BoNt/A has been used in clinical settings as follows:

(1) about 75-125 units of BOTOX®¹ per intramuscular injection (multiple muscles) to treat cervical dystonia;

(2) 5-10 units of BOTOX® per intramuscular injection to treat glabellar lines (brow furrows) (5 units injected intramuscularly into the procerus muscle and 10 units injected intramuscularly into each corrugator supercilii muscle);

(3) about 30-80 units of BOTOX® to treat constipation by intrasphincter injection of the puborectalis muscle;

(4) about 1-5 units per muscle of intramuscularly injected BOTOX® to treat blepharospasm by injecting the lateral pre-tarsal orbicularis oculi muscle of the upper lid and the lateral pre-tarsal orbicularis oculi of the lower lid.

(5) to treat strabismus, extraocular muscles have been injected intramuscularly with between about 1-5 units of BOTOX®, the amount injected varying based upon both the size of the muscle to be injected and the extent of muscle paralysis desired (i.e. amount of diopter correction desired).

(6) to treat upper limb spasticity following stroke by intramuscular injections of BOTOX® into five different upper limb flexor muscles, as follows:

(a) flexor digitorum profundus: 7.5 U to 30 U

(b) flexor digitorum sublimus: 7.5 U to 30 U

(c) flexor carpi ulnaris: 10 U to 40 U

(d) flexor carpi radialis: 15 U to 60 U

(e) biceps brachii: 50 U to 200 U. Each of the five indicated muscles has been injected at the same treatment session, so that the patient receives from 90 U to 360 U of upper limb flexor muscle BOTOX® by intramuscular injection at each treatment session.

Pure botulinum toxin is so labile that it is has limited practical utility to prepare a pharmaceutical composition. Furthermore, the botulinum toxin complexes, such as the toxin type A complex are also extremely susceptible to denaturation due to surface denaturation, heat, and alkaline conditions. Inactivated toxin forms toxoid proteins which may be immunogenic. The resulting antibodies can render a patient refractory to toxin injection.

As with enzymes generally, the biological activities of the botulinum toxins (which are intracellular peptidases) are dependant, at least in part, upon their three dimensional conformation. Thus, botulinum toxin type A is detoxified by heat, various chemicals surface stretching and surface drying. Additionally, it is known that dilution of the toxin complex obtained by the known culturing, fermentation and purification to the much, much lower toxin concentrations used for pharmaceutical composition formulation results in rapid detoxification of the toxin unless a suitable stabilizing agent is present. Dilution of the toxin from milligram quantities to a solution containing nanograms per milliliter presents significant difficulties because of the rapid loss of specific toxicity upon such great dilution. Since the toxin may be used months or years after the toxin containing pharmaceutical composition is formulated, the toxin must be stabilized with a stabilizing agent. To date, the only successful stabilizing agent for this purpose has been the animal derived proteins albumin and gelatin. And as indicated, the presence of animal derived proteins in the final formulation presents potential problems in that certain stable viruses, prions, or other infectious or pathogenic compounds carried through from donors can contaminate the toxin.

Furthermore, any one of the harsh pH, temperature and concentration range conditions required to lyophilize (freeze-dry) or vacuum dry a botulinum toxin containing pharmaceutical composition into a toxin shipping and storage format (ready for use or reconstitution by a physician) can detoxify the toxin. Thus, animal derived or donor pool proteins such as gelatin and serum albumin have been used with some success to stabilize botulinum toxin.

A commercially available botulinum toxin containing pharmaceutical composition is sold under the trademark BOTOX® (available from Allergan, Inc., of Irvine, Calif.). BOTOX® consists of a purified botulinum toxin type A complex, albumin, and sodium chloride packaged in sterile, vacuum-dried form. The botulinum toxin type A is made from a culture of the Hall strain of Clostridium botulinum grown in a medium containing N-Z amine and yeast extract. The botulinum toxin type A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 microns) prior to vacuum-drying. BOTOX® can be reconstituted with sterile, non-preserved saline prior to intramuscular injection. Each vial of BOTOX® contains about 100 units (U) of Clostridium botulinum toxin type A complex, 0.5 milligrams of human serum albumin and 0.9 milligrams of sodium chloride in a sterile, vacuum-dried form without a preservative.

To reconstitute vacuum-dried BOTOX® sterile normal saline without a preservative; 0.9% Sodium Chloride injection is used by drawing up the proper amount of diluent in the appropriate size syringe. Since BOTOX® is denatured by bubbling or similar violent agitation, the diluent is gently injected into the vial. BOTOX® should be administered within four hours after reconstitution. During this time period, reconstituted BOTOX® is stored in a refrigerator (2° to 8° C.). Reconstituted BOTOX® is clear, colorless and free of particulate matter. The vacuum-dried product is stored in a freezer at or below −5° C. BOTOX® is administered within four hours after the vial is removed from the freezer and reconstituted. During these four hours, reconstituted BOTOX® can be stored in a refrigerator (2° to 8° C.).

It has been reported that a suitable alternative to albumin as a botulinum toxin stabilizer may be another protein or alternatively a low molecular weight (non-protein) compound. Carpender et al., Interactions of Stabilizing Additives with Proteins During Freeze-Thawing and Freeze-Drying, International Symposium on Biological Product freeze-Drying and Formulation, 24-26 October 1990; Karger (1992), 225-239.

Many substances commonly used as carriers and bulking agents in pharmaceutical compositions have proven to be unsuitable as albumin replacements in the neurotoxin containing pharmaceutical composition. For example, the disaccharide cellobiose has been found to be unsuitable as a toxin stabilizer. Thus, it is known that the use of cellobiose as an excipient in conjunction with albumin and sodium chloride results in a much lower level of toxicity (10% recovery) after lyophilization of crystalline botulinum toxin type A with these excipients, as compared to the toxicity after lyophilization with only albumin (>75% to >90% recovery). Goodnough et al., Stabilization of Botulinum Toxin Type A During Lyophilization, App & Envir. Micro. 58 (10) 3426-3428 (1992).

Furthermore, saccharides, including polysaccharides, are in general poor candidates to serve as protein stabilizers. Thus, it is known that a pharmaceutical composition containing a protein active ingredient is inherently unstable if the protein formulation comprises a saccharide (such as glucose or a polymer of glucose) or carbohydrates because proteins and glucose are known to interact together and to undergo the well-described Maillard reaction, due to the reducing nature of glucose and glucose polymers. Much work has been dedicated to mostly unsuccessful attempts at preventing this protein-saccharide reaction by, for example, reduction of moisture or use of non-reducing sugars. Significantly, the degrative pathway of the Maillard reaction can result in a therapeutic insufficiency of the protein active ingredient. A pharmaceutical formulation comprising protein and a reducing saccharide, carbohydrate or sugar, such as a glucose polymer, is therefore inherently unstable and cannot be stored for a long period of time without significant loss of the active ingredient protein's desired biological activity.

Notably, one of the reasons human serum albumin can function effectively as a stabilizer of a protein active ingredient in a pharmaceutical composition is because, albumin, being a protein, does not undergo the Maillard reaction with the protein active ingredient in a pharmaceutical composition. Hence, one would expect to find and to look for a substitute for albumin amongst other proteins.

Finding an appropriate substitute for albumin as a stabilizer of the botulinum toxin present in a pharmaceutical composition is difficult and problematic because albumin is believed to function in a pharmaceutical composition as more than a mere bulking agent. Thus, albumin apparently can interact with botulinum toxin so as to increase the potency of the neurotoxin. For example, it is known that bovine serum albumin can act as more than a mere stabilizing excipient for botulinum toxin type A, since bovine serum albumin apparently also accelerates the rate of catalysis of synthetic peptide substrates, which substrates resemble the SNAP-25 intraneuronal substrate for botulinum toxin type A Schmidt, et al., Endoproteinase Activity of Type A Botulinum Neurotoxin Substrate Requirements and Activation by Serum Albumin, J. of Protein Chemistry, 16 (1), 19-26 (1997). Thus, albumin may have a potentiating effect, apparently by affecting rate kinetics, upon the intracellular proteolytic action of a botulinum toxin upon the toxin's substrate. This potentiating effect may be due to albumin which has accompanied the botulinum toxin upon endocytosis of the toxin into a target neuron or the potentiating effect may be due to the pre-existing presence cytoplasmic albumin within the neuron protein prior to endocytosis of the botulinum toxin.

The discovery of the presence of a kinetic rate stimulatory effect by bovine serum albumin upon the proteolytic activity of botulinum toxin type A renders the search for a suitable substitute for albumin in a botulinum toxin containing pharmaceutical formulation especially problematic. Thus, an albumin substitute with desirable toxin stabilization characteristics may have an unknown and possibly deleterious effect upon the rate of substrate catalysis by the toxin, since at least with regard to bovine serum albumin the two characteristics (toxin stabilization and toxin substrate catalysis potentiation) are apparently inherent to the same albumin excipient. This potentiating effect of albumin shows that albumin does not act as a mere excipient in the formulation and therefore renders the search for a suitable substitute for albumin more difficult.

Additionally there are many unique characteristics of botulinum toxin and its formulation into a suitable pharmaceutical composition which constrain and hinder and render the search for a replacement for the albumin used in current botulinum toxin containing pharmaceutical formulations very problematic. Examples of four of these unique characteristics follow.

First, botulinum toxin is a relatively large protein for incorporation into a pharmaceutical formulation (the molecular weight of the botulinum toxin type A complex is 900 kD) and is therefore is inherently fragile and labile. The size of the toxin complex makes it much more friable and labile than smaller, less complex proteins, thereby compounding the formulation and handling difficulties if toxin stability is to be maintained. Hence, an albumin replacement must be able to interact with the toxin in a manner which does not denature, fragment or otherwise detoxify the toxin molecule or cause disassociation of the non-toxin proteins present in the toxin complex.

Second, as the most lethal known biological product, exceptional safety, precision, and accuracy is called for at all steps of the formulation of a botulinum toxin containing pharmaceutical composition. Thus, a preferred potential albumin replacer should not itself be toxic or difficult to handle so as to not exacerbate the already extremely stringent botulinum toxin containing pharmaceutical composition formulation requirements.

Third, since botulinum toxin was the first microbial toxin to be approved for injection for the treatment of human disease, specific protocols had to be developed and approved for the culturing, bulk production, formulation into a pharmaceutical and use of botulinum toxin. Important considerations are toxin purity and dose for injection. The production by culturing and the purification must be carried out so that the toxin is not exposed to any substance that might contaminate the final product in even trace amounts and cause undue reactions in the patient. These restrictions require culturing in simplified medium without the use of animal meat products and purification by procedures not involving synthetic solvents or resins. Preparation of toxin using enzymes, various exchangers, such as those present in chromatography columns and synthetic solvents can introduce contaminants and are therefore excluded from preferred formulation steps. Furthermore, botulinum toxin type A is readily denatured at temperatures above 40 degrees C., loses toxicity when bubbles form at the air/liquid interface, and denatures in the presence of nitrogen or carbon dioxide.

Fourth, particular difficulties exist to stabilize botulinum toxin type A, because type A consists of a toxin molecule of about 150 kD in noncovalent association with nontoxin proteins weighing about 750 kD. The nontoxin proteins are believed to preserve or help stabilize the secondary and tertiary structures upon which toxicity is dependant. Procedures or protocols applicable to the stabilization of nonproteins or to relatively smaller proteins are not applicable to the problems inherent with stabilization of the botulinum toxin complexes, such as the 900 kD botulinum toxin type A complex. Thus while from pH 3.5 to 6.8 the type A toxin and non toxin proteins are bound together noncovalently, under slightly alkaline conditions (pH>7.1) the very labile toxin is released from the toxin complex. Because of its lability, pure toxin has no or limited utility for medical administration.

In light of the unique nature of botulinum toxin and the requirements set forth above, the probability of finding a suitable albumin replacement for the albumin used in current botulinum toxin containing pharmaceutical compositions must realistically be seen to approach zero. Prior to the present invention, only the animal derived proteins, albumin and gelatin, had been known to have utility as suitable stabilizers of the botulinum toxin present in a pharmaceutical formulation. Thus, albumin, by itself or with one or more additional substances such as sodium phosphate or sodium citrate, is known to permit high recovery of toxicity of botulinum toxin type A after lyophilization. Unfortunately, as already set forth, albumin, as a pooled blood product, can, at least potentially, carry infectious or disease causing elements when present in a pharmaceutical composition. Indeed, any animal product or protein such as gelatin can also potentially contain pyrogens or other substances that can cause adverse reactions upon injection into a patient.

Chinese patent application CN 1215084A discusses an albumin free botulinum toxin type A formulated with gelatin, an animal derived protein. U.S. Pat. No. 6,087,327 also discloses a composition of botulinum toxin types A and B formulated with gelatin. These formulations therefore do not eliminate the risk of transmitting an animal protein derived or accompanying infectious element.

Hydroxyethyl Starch

A polysaccharide can be made up of hundreds or even thousands of monosaccharide units held together by glycoside (ether) linkages. Two important polysaccharides are cellulose and starch. Cellulose is the chief structural material in plants, giving plants their rigidity and form. Starch makes up the reserve food supply of plants and is found mainly in various seeds and tubers.

Starch occurs as granules whose size and shape are characteristic of the plant from which the starch is obtained. In general about 80% of starch is a water insoluble fraction called amylopectin. Amylopectin is made up of chains of D-glucose (as glucopyranose) units, each unit being joined by an alpha glycoside linkage to C-4 of the next glucose unit. Like starch, cellulose is also made up of chains of D-glucose units, where each unit is joined by a glucoside linkage to the C-4 of the next unit. Unlike starch though, the glycoside linkages in cellulose are beta linkages. Treatment of cellulose with sulfuric acid and acetic anhydride yields the disaccharide cellobiose. As previously set forth, attempts to stabilize botulinum toxin using cellobiose have been unsuccessful.

A particular starch derivative which can be obtained by treating starch with pyridine and ethylene chlorohydrin, is 2-hydroxyethyl starch, also called hetastarch. U.S. Pat. No. 4,457,916 discloses a combination of a nonionic surfactant and hydroxyethyl starch to stabilize aqueous solutions of tumor necrosis factor (TNF). Additionally, a 6% aqueous solution of 2-hydroxyethyl starch (hetastarch) (available from Du Pont Pharma, Wilmington, Del. under the trade name HESPAN®, 6% hetastarch in 0.9% sodium chloride injection) is known. Albumin is known to act as a plasma volume expander upon intravenous administration to a patient. HESPAN® has also been administrated to patients to achieve a plasma volume expansion effect and in that sense intravenous HESPAN® can be considered a replacement for intravenous albumin.

Hetastarch is an artificial colloid derived from a waxy starch composed almost entirely of amylopectin. Hetastarch can be obtained by introducing hydroxyethyl ether groups onto glucose units of the starch, and the resultant material can then be hydrolyzed to yield a product with a molecular weight suitable for use as a plasma volume expander. Hetastarch is characterized by its molar substitution and also by its molecular weight. The molar substitution can be approximately 0.75, meaning that hetastarch is etherified to the extent that for every 100 glucose units of hetastarch there are, on average, approximately 75 hydroxyethyl substituent groups. The average molecular weight of hetastarch is approximately 670 kD with a range of 450 kD to 800 kD and with at least 80% of the polymer units falling within the range of 20 kD to 2,500 kD. Hydroxyethyl groups are attached by ether linkages primarily at C-2 of the glucose unit and to a lesser extent at C-3 and C-6. The polymer resembles glycogen, and the polymerized D-glucose units are joined primarily by α-1,4 linkages with occasional α-1,6 branching linkages. The degree of branching is approximately 1:20, meaning that there is an average of approximately one α-1,6 branch for every 20 glucose monomer units. Hetastarch is comprised of more than 90% amylopectin.

The plasma volume expansion produced by HESPAN® can approximate that obtained with albumin. Hetastarch molecules below 50 kD molecular weight are rapidly eliminated by renal excretion and a single dose of approximately 500 mL of HESPAN® (approximately 30 g) results in elimination in the urine of approximately 33% of the administered HESPAN® within about 24 hours. The hydroxyethyl group of hydroxyethyl starch is not cleaved in vivo, but remains intact and attached to glucose units when excreted. Significant quantities of glucose are not produced as hydroxyethylation prevents complete metabolism of the smaller hydroxyethyl starch polymers Cellulose can likewise be converted to a hydroxyethyl cellulose. The average molecular weight of 2-hydroxyethyl cellulose (a 2-hydroxyethyl ether of cellulose) is about 90 kD. Unfortunately, hydroxyethyl cellulose, unlike hydroxyethyl starch, is highly reactive and therefore unsuited for use as a stabilizer of a protein active ingredient in a pharmaceutical formulation.

Veterinary Care

Animals may suffer from maladies and injuries that are painful and difficult to treat. For example, some canine breeds, such as German Shepherds, commonly suffer from hip displasia, and may require corrective surgery, such as hip replacement. Horses may suffer from gastrointestinal disorders, such as colic, that may result in significant pain. Similarly, race horses frequently suffer from fractured or broken bones. Horses may also suffer flexor tendon and suspensory ligament injury. Ponies are particularly susceptible to founder, a painful crippling disorder specific to horses.

Additionally, animals may be subjected to painful and irreversible procedures, such as denervation, to modify involuntary natural behaviors, for example ear and tail twitching, in show horses for example. Such drastic procedures are performed for cosmetic reasons. No other humane remedy currently exists to correct such undesirable traits. These procedures permanently damage the animals.

Animals may also cause severe injury to themselves due to what would be a mild condition in humans. Wounds are often exacerbated by stall rubbing in horses resulting in insufficient healing and chronic sores. Sores and skin irritation can result in serious infection in this manner. Other maladies not seen in human patients exist in animals. For example, flies and parasites cause discomfort and act as vectors for disease. Dogs often require hospitalization and or restraint due to biting and scratching related to fleas and mange. Collars are often placed around the animal's neck to prevent the animal from scratching ears affected by mites or surgical procedures. These maladies, if mediated for a time sufficient for the affected area to heal or the parasite to be removed, can be avoided, relieving the animal of suffering and symptoms.

A significant barrier in treating animals and promoting their recovery from injury or surgery is due to an inability to instruct the animal about proper rest and rehabilitation procedures. The difficulty of immobilizing an animal may result in re-injury, self-injury, and/or improper healing. Minor injuries often become catastrophic due to the difficulty of immobilizing animals during and after treatment. Many times the animal must be destroyed. In addition, wild animals (e.g., non-domestic animals) that pose a danger to others or themselves may need to be immobilized to safely return the animal to its native environment. Typically, these animals are tranquilized and moved, or are killed.

Another problem when treating animals arises from the difficulty in administering proper dosages. Animals do not readily swallow oral dosage forms of medicine and often offer violent resistance to injections. Frequent application of short-acting drugs may therefore not be possible. Accordingly, as opposed to humans, animals may require long-acting, single injection pharmaceuticals to receive adequate benefit from the drug.

While pharmaceutical compositions containing human protein may not present problems from a single administration, except in certain individuals or species, repeated dosing may initiate immune responses in animals, including anaphylactic shock. As discussed above, current formulations of botulinum toxin include human serum albumin (HSA) as a stabilizing excipient. These formulations may present serious problems for veterinary use because the presence of a human protein may induce immunogenic responses in other animal species, limiting the efficacy, utility, and safety of the drug. Formulations which do not contain HSA as an excipient, however, should not produce these antibodies. The lack of suitable formulations of botulinum toxin for use in treating non-human animals has impeded the development of methods of using botulinum toxin in veterinary medicine.

Indeed, the majority, if not all, of research regarding the therapeutic aspects of botulinum toxin has focused on the use of botulinum toxin for treating human diseases or ailments. As a necessary precursor, botulinum toxin has experimentally been administered to animals to examine its safety and efficacy in animal models of human diseases. For example, botulinum toxin type B has been administered to mice to treat thermal hyperalgesia (Tsuda et al., (1999) “In vivo pathway of thermal hyperalgesia by intrathecal administration of β-methylene ATP in mouse spinal cord: Involvement of the glutamate-NMDA receptor system”, Br. J. Pharmacol., 127(2):449-456). In addition, botulinum toxin type A has been:

administered to piglets to relieve lower esophageal sphincter (LES) pressure (U.S. Pat. No. 5,437,291, entitled “Method for treating gastrointestinal muscle disorders and other smooth muscle dysfunction”);

injected into gastric wall of rats to treat obesity (Gui et al., (2000) “Botulinum toxin injected in the gastric wall reduces body weight and food intake in rats”, Aliment Pharmacol Ther., 14(6):829-834);

used to assess the efficacy of the neurotoxin to produce better scar formation from facial cosmetic surgery in non-human primates (Gassner et al., (2000) Plast. Reconst. Surg., 105(6):1948-1953);

administered to rats to examine its ability to reduce pain (Aoki et al., (2000) “Methods for treating pain”, U.S. Pat. No. 6,113,915;

administered to rats and dogs to alter vocal cord dynamics for treating spasmodic dysphonia and vocal cord paralysis in humans (Inagi et al., (1998) “Physiologic assessment of botulinum toxin effects in the rat larynx”, Laryngoscope, 108(7):1048-1054; and Cohen and Thompson (1987) “Use of botulinum toxin to lateralize true vocal cords: a biochemical method to relieve bilateral abductor vocal cord paralysis”, Ann. Otol. Rhinol. Laryngol., 96(5):534-41); and

administered to the obicularis oculi muscle of the guinea pig to examine the efficacy of the toxin to treat eye lid spasms (Horn et al., (1993) “Botulinum toxin paralysis of the orbicularis oculi muscle. Types and time course of alterations in muscle structure, physiology and lid kinematics”, Exp. Brain Res., 96(1):39-53).

In addition, botulinum toxin has been administered to rabbits to assess the immunogenicity of the toxin when conjugated with HSA.

Because the administration of botulinum toxin to animals has only been examined experimentally as a model for treating humans with botulinum toxin, researchers have not been concerned with long-term effects of the neurotoxin on animals. In particular, the art has not addressed the immunogenicity of the botulinum toxin compositions in the non-human animals receiving the neurotoxin.

What is needed therefore is a neurotoxin composition with reduced immunogenicity for use in veterinary care, and convenient and effective methods for immobilizing or treating non-human animals.

### SUMMARY

The present invention meets needs specific to animals and in some embodiments provides a replacement for the albumin present in a pharmaceutical composition by a compound which can which can stabilize the botulinum toxin present in the pharmaceutical composition. The albumin replacement compound has the characteristic of low, and preferably negligible, immunogenicity when injected into a patient. Additionally, the preferred albumin replacer has a rapid rate of clearance from the body after injection of the pharmaceutical composition.

Definitions

As used herein, the words or terms set forth below have the following definitions.

The word “about” means that the item, parameter or term so qualified encompasses a range of plus or minus ten percent above and below the value of the stated item, parameter or term.

The phrase “amino acid” includes polyamino acids.

The word “polysaccharide” means a polymer of more than two saccharide molecule monomers, which monomers can be identical or different.

As used herein, “Clostridial neurotoxin” is defined as a neurotoxin produced from, or native to, a Clostridial bacterium, such as Clostridium botulinum, Clostridium butyricum or Clostridium beratti.

As used herein, “botulinum toxin” is defined as a neurotoxin produced by Clostridium botulinum. Botulinum toxin, as used herein, encompasses botulinum toxin serotypes A, B, C₁, D, E, F, or G. Botulinum toxin, as used herein, also encompasses botulinum toxin complexes and purified botulinum toxin. “Purified botulinum toxin” is defined as a botulinum toxin that is isolated, or substantially isolated, from other proteins, including proteins that form a botulinum toxin complex. A purified botulinum toxin may be greater than 95% pure, and preferably is greater than 99% pure.

As used herein, “modified botulinum toxin” is defined as a botulinum toxin that has had at least one of its amino acids deleted, modified, or replaced, as compared to a native botulinum toxin. Additionally, the modified botulinum toxin can be a recombinantly produced neurotoxin, or a derivative or fragment of a recombinantly made neurotoxin. A modified botulinum toxin retains at least one biological activity of the native botulinum toxin, such as, the ability to bind to a botulinum toxin receptor, or the ability to inhibit neurotransmitter release from a neuron. One example of a modified botulinum toxin is a botulinum toxin that has a light chain from one botulinum toxin serotype (such as serotype A), and a heavy chain from a different botulinum toxin serotype (such as serotype B). Another example of a modified botulinum toxin is a botulinum toxin coupled to a neurotransmitter, such as substance p.

The phrase pharmaceutical “composition” means a formulation in which an active ingredient is a neurotoxin, such as a Clostridial neurotoxin. The word “formulation” means that there is at least one additional ingredient in the pharmaceutical composition besides the neurotoxin active ingredient. A pharmaceutical composition is therefore a formulation which is suitable for diagnostic or therapeutic administration (i.e. by intramuscular or subcutaneous injection) to a subject, such as a patient. The pharmaceutical composition can be: in a lyophilized or vacuum dried condition; a solution formed after reconstitution of the lyophilized or vacuum dried pharmaceutical composition with saline or water, or; as a solution which does not require reconstitution. The neurotoxin active ingredient can be one of the botulinum toxin serotypes A, B, C₁, D, E, F, or G or a tetanus toxin, all of which are made by Clostridial bacteria.

The phrase “therapeutic formulation” means a formulation can be used to treat and thereby alleviate a disorder or a disease, such as a disorder or a disease characterized by hyperactivity (i.e. spasticity) of a peripheral muscle.

The words “stabilizing”, “stabilizes”, or “stabilization” mean that upon reconstitution with saline or water of a lyophilized, or vacuum dried botulinum toxin containing pharmaceutical composition which has been stored at or below about −2 degrees C. for between six months and four years, or for an aqueous solution botulinum toxin containing pharmaceutical composition which has been stored at between about 2 degrees and about 8 degrees C for from six months to four years, the botulinum toxin present in the reconstituted or aqueous solution pharmaceutical composition has greater than about 20% and up to about 100% of the toxicity that the biologically active botulinum toxin had prior to being incorporated into the pharmaceutical composition.

Accordingly, as used herein, a “protein stabilizer” is a chemical agent that preserves the biological structure and/or biological activity of a protein. Stabilizers may be proteins or polysacharides. Examples of protein stabilizers include hydroxyethyl starch (hetastarch), serum albumin, gelatin, and collagen. As disclosed herein, the stabilizer may be a synthetic agent that would not produce an immunogenic response in a subject receiving a composition containing the stabilizer. In other embodiments of the invention, the protein stabilizers may be proteins from the same species of animal that is being administered the protein. Additional stabilizers may also be included in a pharmaceutical composition. These additional stabilizers may be used alone or in combination with primary stabilizers, such as proteins and polysaccharides. Secondary stabilizers include, but are not limited to, N-Acetyl-tryptophan, sodium caprylate, amino acids, and divalent cations such as zinc. These compositions may include preservative agents such as benzyl alcohol, benzoic acid, phenol, parabens and sorbic acid. The compositions may be liquid or solid, for example vacuum-dried. The ingredients may be included in a single composition or as a two-component system, for example a vacuum-dried composition reconstituted with a diluent such as saline. This provides the added benefit of allowing incorporation of ingredients which are not sufficiently compatible for long-term shelf storage. For example, the reconstitution vehicle or diluent may include a preservative which provides sufficient protection against microbial growth for the use period, for example one-week of refrigerated storage, but is not present during the two-year freezer storage period during which time it might degrade the toxin. Other ingredients, which may not be compatible with the toxin or other ingredients for long periods of time, may be incorporated in this manner; that is, added in a second vehicle at the approximate time of use.

As used herein, “administration”, or “to administer”, is defined as a step of giving a composition to a subject. The compositions disclosed herein are preferably “locally administered”, i.e., the administration excludes systemic routes of administration. Accordingly, types of local administration include, but are not limited to, intramuscular (i.m.) administration, intradermal administration, subcutaneous administration, intrathecal administration, intraperitoneal (i.p.) administration, administration as a suppository, or as a topical contact, or the implantation of a slow-release device such as a miniosmotic pump. In certain aspects of the invention, the compositions may be administered to the central nervous system, such as by intrathecal administration. In other aspects of the invention, the composition may be administered peripherally or locally, such as by intradermal, intramuscular, or subcutaneous administration.

As used herein, “immobilizing” is defined as a step that prevents a subject from moving one or more body parts. If a sufficient number of body parts are immobilized, the subject will accordingly be immobilized. Thus, “immobilizing” encompasses the immobilization of a body part, such as a limb, and/or the complete immobilization of a subject.

As used herein, “patient” is defined as a human or non-human subject receiving medical care. Accordingly, as disclosed herein, the compositions may be used in treating any animal, such as mammals.

As used herein, a “domesticated animal” is an animal that has adapted to human habitats. Domesticated animals, as defined herein, are tame animals. Accordingly, animals that live with humans, such as pets, are domesticated animals. Farm animals are also domesticated animals as disclosed herein. Laboratory animals are not domesticated animals in reference to the disclosure herein. Humans are not domesticated animals.

In one aspect of the invention, a method for immobilizing a mammal, comprises the step of administering a composition, which comprises at least one botulinum toxin serotype and a polysacharide that stabilizes the botulinum toxin and is nonimmunogenic to the mammal. In one embodiment, the foregoing method may be practiced by administering a composition comprising a hetastarch.

In another embodiment of the invention, a method for immobilizing a mammal, comprises the step of administering a composition to the mammal, wherein the composition comprises (i) at least one botulinum toxin serotype, and (ii) a polysacharide, which comprises a plurality of linked glucopyranose units that each have a plurality of hydroxyl groups present on each of the glucopyranoses present in the polysacharide are substituted, through an ether linkage, with a compound of the formula (CH₂)ₙ—OH, where n can be an integer from 1 to 4.

In another embodiment of the invention, a method for immobilizing a mammal, comprises the step of administering a composition to the mammal, wherein the composition comprises a botulinum toxin, and a hydroxyethyl starch, thereby immobilizing the mammal.

In practicing the foregoing methods, the mammal may be a non-human animal.

In another embodiment of the invention, a method for treating a domesticated animal comprises the step of administering botulinum toxin to the animal. The botulinum toxin may be administered in a pharmaceutical composition. Preferably, the botulinum toxin is administered to the animal in a composition that has a low immunogenicity thereby reducing, and preferably preventing, the development of immunity by the animal to the botulinum toxin. The botulinum toxin may be any one of the seven serotypes of botulinum toxin, or a recombinantly synthesized botulinum toxin. The botulinum toxin may be administered as an acute treatment, or it may be administered chronically.

In another embodiment of the invention, a method for treating a domesticated animal, comprises the step of administering at least one botulinum toxin serotype and a polysacharide that stabilizes the botulinum toxin, to the domesticated animal. In one embodiment, the polysacharide is a hydroxyethyl starch.

In certain embodiments of the invention, the administration of the neurotoxin composition may reduce pain experienced by the animal. In additional embodiments, the animal receiving the neurotoxin may be injured, and the foregoing method promotes the animal's recovery from the injury. One example of an injury that benefits from the invention is a leg injury, such as a broken bone. In practicing the foregoing methods, the compositions disclosed herein may be administered to the injured body part.

The foregoing methods may also be useful in helping a mammal recover from surgery. One example of a surgical procedure is hip displasia surgery. Another example is surgery for a broken bone.

The foregoing methods may be practiced utilizing a composition that comprises a botulinum toxin type A. In other embodiments of the invention, the foregoing methods may be practiced with a composition that comprises botulinum toxin type B. In further embodiments of the invention, the methods may be practiced with a composition that comprises a plurality of botulinum toxin serotypes, such as botulinum toxin serotypes selected from the group consisting of botulinum toxin serotypes A, B, C₁, D, E, F, and G. In certain embodiments of the invention, purified botulinum toxins may be used. In other embodiments, modified botulinum toxins may be used. The compositions used in the foregoing methods may also include one or more amino acids in addition to the botulinum toxin and the polysacharide.

In yet additional embodiments of the invention, the compositions used in the foregoing methods may be administered intramuscularly to the patient. In other embodiments, the compositions may be administered subcutaneously and/or intrathecally.

### DESCRIPTION

The present invention is based upon the discovery that a stable neurotoxin containing pharmaceutical composition can be formulated free of any animal derived protein or donor pool albumin by incorporating a polysaccharide and/or an amino acid into the pharmaceutical composition. In particular, the present invention is based upon the discovery that a stable botulinum toxin containing pharmaceutical composition suitable for administration to a patient for therapeutic effects can be made by replacing the donor pool albumin present in known botulinum toxin containing pharmaceutical compositions with a high molecular weight polysaccharide derived from starch and/or with certain reactive amino acids.

Surprisingly, I have found that a suitable replacement for albumin is a compound which is neither another protein, nor a low molecular weight, non-protein compound. Thus, I have discovered that particular high molecular weight polysaccharides can function as neurotoxin stabilizers in a pharmaceutical composition. As set forth below, an amino acid can also, or in the alternative, be added to the pharmaceutical composition to increase the stability and useful storage life of the pharmaceutical composition.

The polysaccharide used in the present invention can impart stability to a neurotoxin active ingredient, such as a botulinum toxin, present in the pharmaceutical composition by: (1) reducing adhesion (commonly referred to as “stickiness”) of the botulinum toxin to surfaces, such as the surfaces of laboratory glassware, vessels, the vial in which the pharmaceutical composition is reconstituted and the inside surface of the syringe used to inject the pharmaceutical composition. Adhesion of the botulinum toxin to surfaces can lead to loss of botulinum toxin and to denaturation of retained botulinum toxin, both of which reduce the toxicity of the botulinum toxin present in the pharmaceutical composition. (2) reducing the denaturation of the botulinum toxin and/or dissociation of the botulinum toxin from other non-toxin proteins present in the botulinum toxin complex, which denaturation and/or dissociation activities can occur because of the low dilution of the botulinum toxin present in the pharmaceutical composition (i.e. prior to lyophilization or vacuum drying) and in the reconstituted pharmaceutical composition. (3) reducing loss of botulinum toxin (i.e. due to denaturation or dissociation from non-toxin proteins in the complex) during the considerable pH and concentration changes which take place during preparation, processing and reconstitution of the pharmaceutical composition.

The three types of botulinum toxin stabilizations provided by the polysaccharide conserve and preserve the botulinum toxin with it native toxicity prior to injection of the pharmaceutical composition.

In addition, I have discovered that the protein stabilizers disclosed herein reduce the immunogenicity of the pharmaceutical compositions, and thereby are useful in treating conditions that might benefit from neurotoxin treatments in human and non-human subjects. Surprisingly, I have discovered a composition that permits administration of a neurotoxin, such as botulinum toxin, to both human and non-human subjects without resulting in a significant immune response. As discussed above, the presence of HSA in the currently available products of botulinum toxin may preclude the ability for veterinarians to administer botulinum toxin as a treatment for animals.

In certain embodiments of the invention, the pharmaceutical compositions of the invention may comprise a plurality of botulinum toxin serotypes. In other words, the composition may include two or more different botulinum toxin serotypes. For example, a composition may include botulinum toxin serotypes A and B. In another embodiment, a composition may include botulinum toxin serotypes A and E. Using a combination of botulinum toxin serotypes will permit caregivers to customize the composition to achieve a desired effect based on the condition being treated. In an additional embodiment of the invention, the composition may comprise a modified botulinum toxin. The modified botulinum toxin will preferably inhibit the release of neurotransmitter from a neuron, but may have a greater or lower potency than the native botulinum toxin, or may have a greater or lower biological effect than the native botulinum toxin. Because the compositions of the invention may be used for relatively long-term treatment of animals, the compositions may be provided in a relatively pure form. In one embodiment, the compositions are of a pharmaceutical grade. In certain embodiments, the clostridial neurotoxin has a greater than 95% purity. In additional embodiments, the clostridial neurotoxin has a purity greater than 99%.

A preferred polysaccharide for use in the present composition comprises a plurality of glucose monomers (mol wt 180) with one or more substituents on a majority of the glucose monomers, so that the preferred polysaccharide has a molecular weight range of between about 20 kD and about 800 kD. Surprisingly, such a polysaccharide can stabilize a neurotoxin component present in a pharmaceutical composition. The present invention excludes from its scope disaccharide oligosaccharides with a weight average molecular weight of less than about 20 kD. The present invention also excludes from its scope cyclic polymers such as the cyclodextrins. The latter two classes of compounds are excluded from the scope of the present invention because the desired stabilization characteristics of the preferred polysaccharide while requiring a relatively high molecular compound (i.e. molecular weight in excess of 20 kD) do not require and indeed can make no use of the small lipophilic cavity characteristic of the cyclodextrins, because the cyclodextrin lipophilic cavity is much smaller in size than the size of the neurotoxins stabilized by the preferred polysaccharides of the present invention. Additionally, the cyclodextrins are low molecular weight compounds comprising only about 6 to 8 glucose monomers.

The present invention also encompasses a method for stabilizing pharmaceutical compositions which contain a clostridial toxin with a polysaccharide. The stabilizing effect is achieved by bringing a clostridial toxin in contact with the polysaccharide. Examples of suitable polysaccharides within the scope of my invention include certain starch and starch derivatives. As noted, the polysaccharide exhibits a stabilizing effect on the clostridial toxin. Furthermore, the effect of the polysaccharide to stabilize a clostridial toxin can be enhanced by the addition of an amino acid.

Unexpectedly, I have discovered that 2-hydroxyethyl starch demonstrates a unique ability to stabilize the botulinum toxin present in a botulinum toxin containing pharmaceutical composition, thereby providing a pharmaceutical composition which is devoid of the potential for harboring a transmissible disease derived from human blood or blood fraction donor pools or animal derived proteins like gelatin.

Thus, I have discovered that the particular high molecular weight polysaccharide, hydroxyethyl starch, can stabilize the toxin during formulation, drying, storage and reconstitution. Preferably, to further stabilize the protein active ingredient, an amino acid is also included in the polysaccharide containing formulation.

The polysaccharide in the pharmaceutical composition is preferably admixed with the clostridial neurotoxin in an amount of about 1 μg of polysaccharide per unit of botulinum toxin to about 10 μg of polysaccharide per unit of botulinum toxin. More preferably the polysaccharide in the pharmaceutical composition is admixed with the clostridial neurotoxin in an amount of about 4 μg of polysaccharide per unit of botulinum toxin to about 8 μg of polysaccharide per unit of botulinum toxin. In a most preferred embodiment, where the polysaccharide is a hydroxyethyl starch, the hydroxyethyl starch in the pharmaceutical composition is preferably admixed with a botulinum toxin type A complex in an amount of about 5 μg of hydroxyethyl starch per unit of botulinum toxin to about 7 μg of hydroxyethyl starch per unit of botulinum toxin. Most preferably, the hydroxyethyl starch in the pharmaceutical composition is admixed with a botulinum toxin type A complex in an amount of about 6 μg of hydroxyethyl starch per unit of botulinum toxin. Since BOTOX® contains about 100 units of botulinum toxin type A complex per vial and the average molecular weight of hydroxyethyl starch is generally regarded as being between about 20 kD and about 2,500 kD, the most preferred concentration of hydroyethyl starch is between about 1×10⁻⁹ moles per unit of botulinum toxin (M/U) to about 2×10⁻¹² moles per unit of botulinum toxin. In another preferred embodiment, for a 100 U botulinum toxin type A complex pharmaceutical composition, about 600 μg of the hydroxyethyl starch and about 1 mg of an amino acid, such as lysine, glycine, histidine or arginine is included in the formulation. Thus, my invention encompasses use of both a polysaccharide and an amino acid, or a polyamino acid to stabilize the neurotoxin active ingredient in the pharmaceutical composition.

Additionally, my invention also encompasses use of a suitable amino acid in a sufficient amount to stabilize the protein active ingredient in a pharmaceutical composition, either in the presence of or to the exclusion of any polysaccharide being present in the formulation. Thus, I have surprisingly discovered that the inclusion of certain amino acids into a neurotoxin containing, pharmaceutical composition formulation can extend the useful shelf life of such a pharmaceutical composition. Thus, my invention encompasses a neurotoxin containing pharmaceutical composition which includes an amino acid and the use of such a pharmaceutical composition. Without wishing to be bound by theory, I can postulate that since a neurotoxin, such as a botulinum toxin, is susceptible to oxidation, due to the presence of disulfide linkages in the toxin complex, the inclusion of an oxidizable amino acid may act to reduce the probability that oxidizers, such as peroxides and free radicals, will react with the neurotoxin. Thus, the likelihood that the oxidizable neurotoxin disulfide linkage will be oxidized by an oxidizer, such as peroxides and free radicals, can be reduced upon inclusion of an amino acid which can act as an oxidative sink, that is as a scavenger for oxidizing compounds. A suitable amino acid is an amino acid which is subject to oxidation. Examples of preferred amino acids are methionine, cysteine, tryptophan and tyrosine. A particularly preferred amino acid is methionine.

A preferred embodiment of my invention can also include the use of two or more amino acids either alone or in combination with a polysaccharide to stabilize the protein active ingredient in a pharmaceutical composition. Thus, for a 100 U botulinum toxin type A containing pharmaceutical composition, about 0.5 mg of lysine and about 0.5 mg of glycine can be used, either with or without between about 500 μg and about 700 μg of hetastarch.

Thus, as set forth above, my invention encompasses a protein containing, pharmaceutical composition which includes a polysaccharide. The polysaccharide acts to stabilize the protein active ingredient in the pharmaceutical composition. Additionally, my invention also includes a protein containing, pharmaceutical composition which includes a polysaccharide and an amino acid. Surprisingly, I have discovered that the inclusion of certain amino acids into a neurotoxin containing, pharmaceutical composition formulation which includes a carbohydrate can extend the useful shelf life of such a pharmaceutical composition. Thus, my invention encompasses a neurotoxin containing pharmaceutical composition which includes both a polysaccharide and an amino acid and the use of such a pharmaceutical composition. Furthermore, my invention also encompasses use of an amino acid without any polysaccharide being present in the protein active ingredient pharmaceutical composition.

It is known that protein containing pharmaceutical compositions which also contain sugars, polysaccharides and/or carbohydrates (referred to hereafter as “reactive compounds”) are inherently unstable due to the fact that a protein and one of the three indicated reactive compounds can undergo the well-described Maillard reaction. Extensive, largely fruitless, research has been carried out to try and reduce the incidence or prevalence of this (for example) protein-polysaccharide Maillard reaction, by reduction of moisture or by the use of non-reducing sugars in the formulation. My discovery is based upon the observation that inclusion of a high concentration of a highly reactive amino acid encourages the Maillard reaction to take place between the stabilizing polysaccharide and the added amino acid. By providing an abundant amine source for the carbohydrate to react with, the probability of the protein drug (i.e. botulinum toxin active ingredient) becoming involved in the Maillard is reduced, thereby reducing this degradation pathway of the protein active ingredient and in this manner thereby stabilizing the protein active ingredient in the pharmaceutical composition.

Preferably, any compound containing a primary or secondary amine can be used for this purpose. Most preferred are amino acids, such as lysine, glycine, arginine. Polyamino acids, such as polylysine are also suitable. Cationic amino acids such as lysine may undergo ionic attraction, binding acidic proteins (e.g., botulinum toxins) and shield the active protein from contact with sugars. Polylysine, in addition to being larger and therefore more likely to act as a shield, provides the additional advantage of being antibacterial.

Another aspect of my invention is to pre-react the sugar and amino acid components to exhaust Maillard reaction potential before adding the active protein component (botulinum toxin) to the sugar and amino acid formulation ingredients, thereby substantially limiting the active protein's exposure to Maillard reactions.

Thus, my invention encompasses a pharmaceutical composition containing and the use of an amino acids and polyamino acids as Maillard reaction inhibitors in protein (i.e. botulinum toxin) drug formulations which contain starches, sugars and/or polysaccharides.

The invention embodies formulations of active proteins (e.g., botulinum toxin) in combination with a stabilizing starch, sugar, or polysaccharide or combination of these, and an amino acid such as lysine.

Significantly, I have discovered that hydroxyethyl starch does not undergo or undergoes a much attenuated rate or level of Maillard reactions with a protein, such as a botulinum toxin, when hydroxyethyl starch is compared to other polysaccharides or carbohydrates. Additionally, I have discovered that inclusion of an amino acid enhances the preservation effect of hydroxyethyl starch, possibly by acting as a competitive inhibitor, that is by competing with the toxin for Maillard reaction reactive sugars. For this purpose, amino acids such as lysine, glycine, arginine and histidine are preferred amino acids. Polyamino acids, such as polylysine, which exhibit the desired competitive inhibition behavior can also be used. Notably, the specified amino and poly amino acids can also exhibit antimicrobial properties, providing therefore the added benefit of reducing bacterial contamination in the pharmaceutical composition.

Reducing sugars, such as glucose and glucose polymers, undergo Maillard reaction with proteins. Even sugar alcohols like mannitol can react, albeit sometimes through contaminants or degradation products. Therefore a polysaccharide can stabilize the toxin for a period of time only to chemically react later, thereby causing reduced storage stability. It is obvious that the choice of polysaccharide is critical. I have discovered that the rate of hydroxyethyl starch participation in the Maillard reaction is very low. Additionally, I have found that hydroxyethyl cellulose, although structurally very similar to hydroxyethyl starch, is unsuitable to use as a stabilizer, since I have found that hydroxyethyl cellulose can rapidly react in a model system with lysine. This not only means that hydroxyethyl starch has an obvious advantage over other sugar (i.e. polysaccharide) stabilizers, but that even excipients similar to hydroxyethyl starch, such as hydroxyethyl cellulose, can be unsuitable to use as stabilizers of a protein active ingredient in a pharmaceutical formulation.

As noted, hydroxyethyl starch, can participate to at least some extent, in Maillard reactions. Thus, and as set forth above, a polysaccharide alone may not be sufficient to provide optimal stabilization of the toxin. Thus, I discovered the advantages of inclusion of an amino acid to act as a competitive inhibitor. Without wishing to be bound by theory, the hypothesis is that by providing another amine source, in high concentrations compared to the toxin, the probability of the Maillard reaction occurring with the toxin is reduced, thereby stabilizing the toxin. Any amino acid can be used however lysine being highly reactive and is a preferred amino acid.

My invention also encompasses addition of a zinc ion source to a botulinum toxin containing pharmaceutical formulation. Metals, especially divalent cations, are reported to greatly influence the success of a freeze-dried formulation due to various cryo-properties including the lattice structures of formed ices. Extraneous metals such as copper and iron species lend themselves to radical oxidations and are generally to be avoided. More specifically Botulinum Type-A toxin is dependent upon bound zinc for activity. Many of the proposed excipients or reaction products of the excipients will chelate metals. This could lead to formation of unstable ices and/or inactivated toxin. By supplying ample zinc in the formulation the presence of desirable divalent cations is assured, the likelihood of zinc loss by the toxin is reduced, and stability enhanced. This can be accomplished by the addition of ZnSO₄ or ZnCl₂ to a botulinum toxin pharmaceutical composition.

It is known to make recombinant human serum albumin (rHSA) production using an expression strain of the methylotrophic yeast Pichia pastoris with high productivity. Botulinum toxin containing pharmaceutical formulations can be prepared containing stabilizing rHSA. rHSA expressed by genetically altered yeast host cells, while having the same primary amino acid sequence, differ in other respects from plasma derived HSA. Thus, while eukaryotic, yeast, lacking many intracellular processes found in mammals. Additionally, pHSA (plasma derived human serum albumin) is made in non-glycosylated form and undergoes extracellular, non-enzymatic addition of glucose. Hence, the carbohydrate moieties of pHSA and rHSA differ. Furthermore, it is known that the amounts of palmitic acid and stearic acid present in rHSA are much lower than those in pHSA. These differences can be expected to result in differences in, inter alia, ligand binding, conformational stability and molecular charge between rHSA and pHSA. It was therefore surprising to discover that rHSA can be used to stabilize botulinum toxin, particularly in light of the known kinetic rate effect of pHSA upon botulinum toxin. An advantage of rHSA is that it is free of blood derived pathogens. Thus, another aspect of my invention encompasses replacement of the blood derived serum albumin in a pharmaceutical composition with recombinant serum albumin. Preferably, the recombinant serum albumin is present in the botulinum toxin containing pharmaceutical formulation with acetyltryptophanate, as set forth below.

Commercially available human serum albumin is heated at 60° C. for ten hours as a requirement to eliminate potentially infectious agents derived from the human blood pool. In order to prevent serious denaturation during this process two stabilizers are added: sodium acetyltryptophanate and sodium caprylate. With a rHSA (or other recombinant serum albumin) there is no need to add these ingredients as no disease risk exists and no heating step is required. I have discovered that addition of sodium acetyltryptophanate to rHSA enhances the thermal stability beyond that shown by the use of sodium caprylate alone, even when the concentration of sodium caprylate is doubled. Without wishing to be bound by theory, I believe that may be due to the caprylate binding to only one site whereas sodium acetyltryptophanate binds two sites. Binding this second site appears to enhance the resistance to thermal perturbation. I can further postulate that the addition of sodium acetyltryptophanate may in some way enhance the stability of botulinum toxin formulations, possibly by maintaining a thermodynamically favorable conformation in the molecule, binding the toxin, or by preventing denaturation of the human serum albumin itself. Therefore the invention discloses the desirability to include secondary stabilizers in protein-stabilized formulations such as HSA or rHSA. These include N-Acetyl-tryptophan (sodium tryptophanate), sodium caprylate, fatty acids, and divalent cations such as zinc (for example zinc chloride).

Although recombinant serum albumin is preferred over animal-derived serum albumin, it is a further aspect of my invention to provide a composition that may comprise a serum albumin obtained from the species of animal intended to be treated. For example, if a horse were to receive an injection of a clostridial neurotoxin, such as botulinum toxin, it may be desirable to utilize a composition comprising the neurotoxin and an equine serum albumin as a stabilizer. Similarly, if a cow were to receive an injection of a clostridial neurotoxin, the composition containing the neurotoxin may include bovine serum albumin as a stabilizer. This reasoning will similarly apply to other animal species, including primate serum albumin for non-human primates, porcine serum albumin for pigs, canine serum albumin for dogs, feline serum albumin for cats, and murine serum albumin for rodents. Other species-specific serum albumins are provided in the compositions of the invention.

One composition of the invention may comprise botulinum toxin type A, and serum albumin from horses, dogs, cats, rabbits, pigs, or rodents.

Another composition of the invention may comprise botulinum toxin type B, C₁, D, E, F, or G; and serum albumin from non-human primates, cows, horses, pigs, dogs, cats, or rodents.

As persons skilled in the art will readily appreciate, although the serum derived albumins may have some of the shortcoming discussed herein, they may still find some use in veterinary care.

My invention also encompasses addition of a preservative, either in the diluent or formulation itself, to allow extended storage. A preferred preservative is preserved saline containing benzyl alcohol.

A liquid formulation can be advantageous. A single-step presentation (e.g., pre-filled syringe) or a product configuration that the user perceives as a single-step presentation (e.g., dual-chambered syringe) would provide convenience by eliminating the reconstitution step. Freeze-drying is a complicated, expensive and difficult process. Liquid formulations are often easier and cheaper to produce. On the other hand liquid formulations are dynamic systems and therefore more susceptible to excipient interaction, fast reactions, bacterial growth, and oxidation than freeze-dried formulations. A compatible preservative might be needed. Anti-oxidants such as methionine might also be useful as scavengers especially if surfactants are used to reduce adsorption as many of these compounds contain or produce peroxides. Any of the stabilizing excipients which can be used in a freeze-dried formulation (e.g., hydroxyethyl starch or an amino acid such, lysine) might be adapted to use in a liquid formulation to assist in reducing adsorption and stabilize the toxin. Suspensions similar to those developed for insulin are also good candidates. Additionally, stabilizing botulinum toxin in a liquid vehicle might require a low pH vehicle as the toxin is reported to be labile above pH 7. This acidity could produce burning and stinging upon injection. A binary syringe could be employed. Inclusion of a co-dispensed buffer, sufficient to raise the pH to physiologic levels, would alleviate injection discomfort of a low pH while maintaining the toxin at a low pH during storage. Another dual-chambered syringe option would include diluent and lyophilized material segregated in separate chamber, only mixing upon use. This option provides the advantages of a liquid formulation without the additional resources and time.

Thus, the botulinum toxin can be prepared at low pH to be co-dispensed with a buffer which raises the pH to at or near physiological pH at the time of administration. The two chamber or binary syringe can have in the first chamber (next to the plunger) a liquid formulation of a botulinum toxin with a pH between 3 to 6 (i.e. at pH 4.0). The second chamber (next to the needle tip) can contain a suitable buffer, such as phosphate buffered saline at a higher pH (i.e. pH 7.0). Alternately, the first chamber can contain a saline diluent and the second chamber can contain a freeze dried or lyophilized neurotoxin formulation. The two chambers can be joined in such a way and the buffering components selected in such a way that the solutions mix at or near the needle, thereby delivering the final solution at a physiological pH. Suitable two chamber syringes to use as pre-filled syringes for the purposes set forth herein can be obtained from Vetter Pharma-Fertigung of Yardley, Pa.

There are distinct advantages to formulating botulinum toxin at a low pH. The toxin has a low isoelectric point (pI) and formulating proteins near their pI is a known way to stabilize a protein. Additionally, the toxin is used at a very low concentration making surface adsorption a problem. Use of a low pH solution can suppress ionization of toxin sites likely to interact with surfaces. The syringe and plunger materials are materials which reduce surface adsorption by the toxin. Suitable such materials are polypropylene.

As discussed herein, the neurotoxin may be prepared and purified using techniques well-known in the art. The purified toxin may subsequently be diluted in a stabilizer such as a polysacharide (e.g., hetastarch), or a recombinant serum albumin, or a serum albumin of the species of animal receiving the neurotoxin. It is preferred that the stabilizer prevents or reduces denaturation of the toxin, and produces no, or minimal, immunogenic responses in the animal that will receive the toxin. Aliquots of the diluted toxin are then lyophilized using conventional procedures.

The lyophilized neurotoxin may be reconstituted before administering the neurotoxin to a subject by adding water, saline, or any buffer solution to the lyophilized neurotoxin. In certain embodiments, sodium free buffers may be preferred to help reduce denaturation of the neurotoxin.

The pharmaceutical compositions of the invention can be administered using conventional modes of administration. In preferred embodiments of the invention, the compositions are administered intramuscularly or subcutaneously to the subject. In other embodiments, the compositions of the invention may be administered intrathecally. In addition, the compositions of the invention may be administered with one or more analgesic or anesthetic agents.

The most effective mode of administration and dosage regimen for the compositions of this invention depends upon the type, severity, and course of the condition being treated, the animal's health and response to treatment, and the judgment of the treating doctor. Accordingly, the methods and dosages of the compositions should be tailored to the individual subject.

By way of example, and not by way of limitation, it may be preferred to administer the composition of the invention intramuscularly to reduce muscle spasms associated with a specific condition. Spasms in veterinary animals can be caused by congenital defects (Hickford et al., J Small Anim Pract 1998 39(6):281-5), hip dysplasia (Bowen, Am J Vet Res 1974 35(5):661-8), inflammation (Anderson and Harvey, J Vet Dent 1993 10(1):6-8) multiple sclerosis, stroke, tumors, traumas, or neurologic degenerative diseases. Administering the compositions disclosed herein to animals may be particularly effective in treating specific conditions such as “teary eyes” or “tail flick” in animals. Injecting botulinum toxin periodically (e.g., every three to six months) in the same areas may allow for long term, indefinite relief of spastic symptoms in these animals without the unwanted development of resistance to the toxin.

Similarly, the compositions of the invention may be administered intramuscularly to immobilize an injured animal. Injuries that result in fractures and tendon rupture require the limb associated with the site of injury to be completely immobilized so as to prevent reinjury. Although the site is placed in a cast, often the animals will reinjure themselves with ongoing activity. The only way to completely immobilize the animal is general anaesthesia, which cannot be employed for long term uses. In addition, immobilization of the injured body part with a cast, for example, may cause bone malformation and with improper functional recovery (Hawthorne et al., J Am Anim Hosp Assoc 1999 35(2):135-146).

Intramuscular injection of the compositions of the invention can result in selective and reversible immobilization of the animal, or body parts thereof. For example, the compositions of the invention may be administered locally to one or more muscle groups of an injured body part. In other cases, it may be necessary to perform whole body immobilization of the musculature involved in whole body movements (limb muscles and abdominal muscles) without impinging the respiratory system or muscles involved in food intake. In such a scenario, the animal will not be able to willfully move any parts of the body involved in ambulatory movements, so physical restraints may not be necessary. Other functions required to maintain the overall health of the animal would preferably be still intact, so therefore a normal functioning animal is kept at a “temporary paralytic” state. Furthermore, depending on the type of neurotoxin in the composition administered to the animal, the rate of recovery may be controlled. For example, a composition containing botulinum toxin type E may be administered if the caregiver decides that a relatively shorter time of paralysis is needed to promote recovery. In addition, because the effects of the neurotoxin wear off gradually, the activity of the immobilized body parts can be regained at a rate which is most desirable to achieve proper healing and recovery.

The compositions of the invention may also be injected into smooth muscles (as compared to striated muscles) to treat colonic, bladder, esophageal, or gastrointestinal dysfunction, including, but not limited to achalasia, anal fissure, hyperactive sphincter of oddi. The administration of the compositions may reduce or prevent unfavorable systemic consequences from treatment with drugs that do not specifically act on the organ of interest.

Compositions containing botulinum toxin may be administered intramuscularly, intrathecally, or subcutaneously to relieve pain experienced by the animal. These treatments are also restricted to the site of injection and have minimal side effects compared to current systemic approaches of treating these pain syndromes with pain relieving drugs.

Pain may be difficult to determine in an animal; however, animals may exhibit one or more behaviors indicative of the animal experiencing pain. For example, the animal may cease its normal activities, or it may favor a potentially injured limb. The animal may stop eating, may whine, may have an increased respiration rate, or heart rate, may have an abnormal temperature, or may appear restless. In addition, animals experience intestinal discomfort, such as colic, may experience one or more of the following symptoms in addition to the symptoms identified above, paw the ground, may sweat, may be restless, may appear bloated, may be experience muscle tremors.

Relief from pain by practicing the methods of the invention may be determined by observing the reduction in the number of symptoms that the animal is exhibiting. One or more of the symptoms may be reduced.

Injection of botulinum toxin to multiple muscles, such as leg muscles, may be effective to cause a potent, prolonged, yet reversible immobilization of wild, non-domesticated animals. This procedure can be used for medical evaluation of the patient, various local or minor medical or dental procedures, or for transport. In practicing these methods, it may be desirable to also inject either before, or at the same time, a tranquilizer to provide immediate effects of immobilization.

As indicated above, dosages of the neurotoxin, such as botulinum toxin, in the compositions may vary. In one embodiment, the compositions contain a therapeutically effective amount of neurotoxin, for example, between about 1 U and about 500 U of botulinum toxin type A. Preferably the amounts are between about 10 U and about 300 U. More preferably the amount is between about 20 U and 250 U, such about 50 U to 200 U, or 70 U.

Alternatively, botulinum toxin, such as botulinum toxin type A, can be administered in amounts between about 10⁻³ U/kg and about 60 U/kg to alleviate pain experienced by a mammal. Preferably, the botulinum toxin used is administered in an amount of between about 10⁻² U/kg and about 50 U/kg. More preferably, the botulinum toxin is administered in an amount of between about 10⁻¹ U/kg and about 40 U/kg. Most preferably, the botulinum toxin is administered in an amount of between about 1 U/kg and about 30 U/kg. In a particularly preferred embodiment of the present disclosed methods, the botulinum toxin is administered in an amount of between about 1 U/kg and about 20 U/kg.

Compositions containing other serotypes of botulinum toxin may contain different dosages of the botulinum toxin. For example, botulinum toxin type B may be provided in a composition at a greater dose than a composition containing botulinum toxin type A. In one embodiment of the invention, botulinum toxin type B may be administered in an amount between about 1 U/kg and 150 U/kg. Botulinum toxin type B may also be administered in amounts of up to 20,000 U (mouse units, as described above). In another embodiment of the invention, botulinum toxin types E or F may be administered at concentrations between about 0.1 U/kg and 150 U/kg. In addition, in compositions containing more than one type of botulinum toxin, each type of botulinum toxin can be provided in a relatively smaller dose than the dose typically used for a single botulinum toxin serotype. The combination of botulinum toxin serotypes may then provide a suitable degree and duration of paralysis without an increase in diffusion of the neurotoxins (e.g., see U.S. Pat. No. 6,087,327).

### EXAMPLES

### Example 1

### Botulinum Toxin Pharmaceutical Composition

As previously set forth, botulinum toxin type A complex can be obtained from a culture of the Hall strain of Clostridium botulinum grown in a medium containing N-Z amine and yeast extract. The botulinum toxin type A complex is purified from the culture solution by a series of acid precipitations to a crystalline complex consisting of the active high molecular weight toxin protein and an associated hemagglutinin protein. The crystalline complex is then re-dissolved in a solution containing saline and albumin and sterile filtered (0.2 microns) prior to vacuum-drying. BOTOX® is then reconstituted with sterile, non-preserved saline prior to intramuscular injection. Each vial of BOTOX® contained about 100 units (U) of Clostridium botulinum toxin type A complex, 0.5 milligrams of human serum albumin and 0.9 milligrams of sodium chloride in a sterile, vacuum-dried form without a preservative. Alternately, the human serum albumin can be replaced by a recombinantly made albumin.

### Example 2

### Botulinum Toxin Pharmaceutical Composition Containing 2-Hydroxyethyl Starch

Botulinum toxin type A purified neurotoxin complex pharmaceutical formulations were prepared in the same manner set forth in Example 1 above, except that the 0.5 milligrams of albumin was replaced by either 500 μg or 600 μg of hetastarch. It was determined that full potency was maintained upon preparation of the hetastarch containing formulations. Thus, with both hetastarch containing formulations, the potency of the albumin-free, hetastarch containing composition, as measured at the time of reconstitution of the lyophilized, 100 U (±20 U) botulinum toxin type A complex, was from 96 to 128 units. Three separate hetastarch, botulinum toxin type A complex pharmaceutical compositions had potency measurements, at the time of reconstitution, of, respectively, 105, 111 and 128 units. Potency was measured using the standard administration to mice toxin potency assay.

### Example 3

### Botulinum Toxin Pharmaceutical Composition Containing Glycine

Botulinum toxin type A purified neurotoxin complex pharmaceutical formulations was prepared in the same manner set forth in Example 1 above, except that the 0.5 milligrams of albumin was replaced by either 500 μg or 600 μg of hetastarch. In addition 1 mg of glycine was added to the formulation. A lyophilized, hetastarch plus glycine, albumin-free, 100 U botulinum toxin type A complex, pharmaceutical composition was then stored for seven months at −5° C. At the end of this seven month period the potency of this hetastarch plus glycine toxin formulation was determined, using the mouse administration assay, to be essentially unchanged (i.e. potency differed by less than 5% from the original potency).

### Example 4

### Botulinum Toxin Pharmaceutical Composition Containing Lysine

100 U botulinum toxin type A purified neurotoxin complex pharmaceutical formulations are prepared in the same manner set forth in Example 1 above, except that the 0.5 milligrams of albumin was replaced by 600 μg of hetastarch. In addition 1 mg of lysine is added to the formulation. A lyophilized, hetastarch plus lysine, albumin-free, 100 U botulinum toxin type A complex, pharmaceutical composition is then stored for one year at −5° C. At the end of this one year period the potency of this hetastarch plus lysine, toxin formulation is determined, using the mouse administration assay, to be essentially unchanged (i.e. potency differs by less than 5% from the original potency).

### Example 5

### Botulinum Toxin Pharmaceutical Composition Containing Histidine

100 U botulinum toxin type A purified neurotoxin complex pharmaceutical formulations are prepared in the same manner set forth in Example 1 above, except that the 0.5 milligrams of albumin was replaced by 600 μg of hetastarch. In addition 1 mg of histidine is added to the formulation. A lyophilized, hetastarch plus histidine, albumin-free, 100 U botulinum toxin type A complex, pharmaceutical composition is then stored for one year at −5° C. At the end of this one year period the potency of this hetastarch plus histidine, toxin formulation is determined, using the mouse administration assay, to be essentially unchanged (i.e. potency differs by less than 5% from the original potency).

### Example 6

### Botulinum Toxin Pharmaceutical Composition Containing Arginine

100 U botulinum toxin type A purified neurotoxin complex pharmaceutical formulations are prepared in the same manner set forth in Example 1 above, except that the 0.5 milligrams of albumin was replaced by 600 μg of hetastarch. In addition 1 mg of arginine is added to the formulation. A lyophilized, hetastarch plus arginine, albumin-free, 100 U botulinum toxin type A complex, pharmaceutical composition is then stored for one year at −5° C. At the end of this one year period the potency of this hetastarch plus arginine, toxin formulation is determined, using the mouse administration assay, to be essentially unchanged (i.e. potency differs by less than 5% from the original potency).

### Example 7

### Botulinum Toxin Pharmaceutical Composition Containing An Amino Acid

Botulinum toxin type A purified neurotoxin complex pharmaceutical formulations can be prepared in the same manner set forth in Example 1 above, except that the 0.5 milligrams of albumin can be replaced by about 1 mg of an amino acid such as lysine, glycine, histidine or arginine. A lyophilized, polysaccharide free, albumin free plus glycine, albumin-free, 100 U botulinum toxin type A complex, pharmaceutical composition can be stored for at least one year −5° C., and at the end of this period can have a potency of which is essentially unchanged (i.e. potency can differ by less than 5% from the original potency).

### Example 8

### Use of a Botulinum Toxin Pharmaceutical Composition

A 48 year old male is diagnosed with a spastic muscle condition, such as cervical dystonia. Between about 10⁻³ U/kg and about 35 U/kg of a botulinum toxin type A pharmaceutical composition containing 600 μg of hetastarch and 1 mg of an amino acid, such as lysine, is injected intramuscularly into the patient. Within 1-7 days the symptoms of the spastic muscle condition are alleviated and alleviation of the symptoms persists for at least from about 2 months to about 6 months.

### Example 9

### Use of a Botulinum Toxin Pharmaceutical Composition to Treat Equine Colic

A two year old thoroughbred is brought into a veterinary clinic for evaluation of a recent onset of poor feeding, fussiness, and being rider-unfriendly. Blood chemistry indicates that the horse has metabolic disturbances of low glucose, hyperkalemia, and low blood count. Urinary excretion indicative of dehydration is noticed. The horse is diagnosed with colic. A composition containing between about 10⁻³ U/kg and about 35 U/kg of botulinum toxin A and 600 μg of hetastarch is intracecally and transabdominally injected at several sites on three separate office visits. After three months, all signs of colic disappear and the horse is reintroduced into the racing circuit.

### Example 10

### Use of a Botulinum Toxin Pharmceutical Composition to Treat a Canine

An owner of a 3 year-old German shepherd comes to a clinic concerned about a week history of limping and general lethargy of her dog. An x-ray is performed and a diagnosis of hip dysplasia is made. The animal undergoes hip replacement surgery. The dog's hips are cast, and botulinum toxin A injection is suggested to provide further immobilization of the rear legs and hips. Multiple injections of a composition comprising about 20 U/kg of botulinum toxin type A and 600 μg of hetastarch are given to the animal. The dog recuperates for about 4 weeks before removal of the cast. After removal of the cast, the dog continues to rest. A little after 8 weeks, the dog begins to regain some function of his legs. After 4 months, the dog has regained enough function to begin regular walking. By 6 months, the dog resumes normal activities.

A year after the hip replacement, the dog is lethargic, does not eat, and appears to be in distress. The dog is brought to a veterinarian for diagnosis. Except for low blood glucose, all other blood chemistry values appear normal. A barium scan of the upper GI finds narrowing of the lower esophageal sphincter, resulting in a megaesophagus. Diagnosis of achalasia is made, and botulinum toxin injection is suggested. An injection of a composition containing botulinum toxin type A and hetastarch into the lower esophageal sphincter muscles is performed using fiberoptic guidance. Importantly, one week later, a second barium scan is performed, revealing a complete opening of the sphincter, and indicating that the dog has not developed immunity to the toxin from his earlier surgery. The owner notices cessation of vomiting and normal feeding habits.

### Example 11

### Use of a Botulinum Toxin Pharmaceutical Composition to Immobilize an Animal

A 4 year old angus bull is transported under general anesthesia to a large animal veterinary clinic facility for an evaluation of a fractured tibia resulting from a fight with another bull. X-ray evaluation reveals 3 fracture points restricted to the right, rear tibia without fractures to the fibula. The shin is plaster-casted, and it is recommended that the bull be completely immobilized to allow the leg to heal properly. A composition containing about 40 U/kg of botulinum toxin A and 600 μg of hetastarch is injected in several large muscle groups, including all the limb front and rear limb muscles. No injection is made to the abdominal rectus, cervical spinal, or facial muscles. The bull is completely immobilized but retains full functional control over food intake. After six months and two complete panels of injections, the limb heals enough to allow weight-bearing, but restricted movements within its own bull pen. After three more months the bull is allowed to be with other bulls to resume more physical activities.

### Example 12

### Use of a Botulinum Toxin Composition to Treat Canine Muscle Spasms

A 10 year old Yorkshire terrier is brought into a clinic for an evaluation of muscle spasms confined to the right half of the body. A CAT scan of the head reveals a large, intraparenchymal mass in the left brain, and biopsy reveals a grade II astrocytoma. Euthanasia is proposed by the veterinarian.

However, the owner still wants to spend the last few weeks of quality time with her dog and does not want the animal to suffer any pain that may result from the muscle spasms. Botulinum toxin injection is suggested. Injections are made in spastic musculature on the right side. The injections successfully abolish all spastic activities. The dog lives the remaining two weeks without spasms and according to the owner, happy and “appeared to be rather healthy”.

### Example 13

### Use of a Botulinum Toxin Pharmaceutical Composition to Treat Founder

A four-year-old Welsh mare has been habitually overfed a rich diet of alfalfa hay. The owner finds the pony hobbling in obvious pain and unable to walk normally. Upon examination the veterinarian diagnoses the problem as founder and recommends a strict diet and regular exercise until the animal's symptoms subside. However, neither the owner nor the veterinarian can convince the pony to walk more than a few steps at a time. If normal exercise is not quickly resumed the animal will be permanently crippled. A composition containing approximately of 100 U botulinum toxin type A and 500μg HSA is injected into the hooves enabling the pony to apply pressure to the previously painful area. Within two weeks the pony regains its normal gait and resumes pulling a small cart containing the owner's children.

A pharmaceutical composition according to the invention disclosed herein has many advantages, including the following:

1. the pharmaceutical composition can be prepared free of any blood product, such as albumin and therefore free of any blood product infectious element such as a prion.

2. the pharmaceutical composition has stability and high % recovery of toxin potency comparable to or superior to that achieved with currently available pharmaceutical compositions.

Accordingly, the pharmaceutical compositions disclosed herein have reduced immunogenicity and therefore enable medical caregivers to treat animals with a reduced risk that the animal will develop an immune response to the administered composition.

Although the present invention has been described in detail with regard to certain preferred methods, other embodiments, versions, and modifications within the scope of the present invention are possible. For example, a wide variety of stabilizing polysaccharides and amino acids are within the scope of the present invention.

Accordingly, the spirit and scope of the following claims should not be limited to the descriptions of the preferred embodiments set forth above.

I claim:

1. A method for immobilizing a mammal, comprising the step of administration of a composition, which comprises at least one botulinum toxin serotype and a polysacharide that stabilizes the botulinum toxin and is nonimmunogenic to the mammal, thereby immobilizing the mammal.

2. The method of claim 1, wherein the mammal is a non-human animal.

3. The method of claim 1, wherein the composition further comprises an amino acid in addition to the botulinum toxin and the polysacharide.

4. The method of claim 1, wherein the composition comprises a plurality of botulinum toxin serotypes.

5. The method of claim 2, wherein the animal is injured.

6. The method of claim 5, wherein the method promotes the animal's recovery from the injury.

7. The method of claim 6, wherein the injury is a leg injury.

8. The method of claim 1, wherein botulinum toxin is administered to an injured body part.

9. The method of claim 1, wherein the mammal is recovering from surgery.

10. The method of claim 9, wherein the surgery was hip displasia surgery.

11. The method of claim 1, wherein the botulinum toxin is botulinum toxin type A.

12. The method of claim 1, wherein the botulinum toxin is botulinum toxin type B.

13. The method of claim 1, wherein the polysacharide is a hetastarch.

14. The method of claim 1, wherein the botulinum toxin is a purified botulinum toxin.

15. The method of claim 1, wherein the botulinum toxin is administered intramuscularly.

16. The method of claim 1, wherein the botulinum toxin is a modified botulinum toxin.

17. A method for immobilizing a mammal, comprising the step of administration of a composition to the mammal, wherein the composition comprises
(i) at least one botulinum toxin serotype, and
(ii) a polysacharide, which comprises a plurality of linked glucopyranose units that each have a plurality of hydroxyl groups present on each of the glucopyranoses present in the polysacharide are substituted, through an ether linkage, with a compound of the formula (CH₂)ₙ—OH, where n can be an integer from 1 to 4,
thereby immobilizing the mammal.

18. A method for immobilizing a mammal, comprising the step of administration of a composition to the mammal, wherein the composition comprises a botulinum toxin, and a hydroxyethyl starch, thereby immobilizing the mammal.

19. A method for treating a domesticated animal, comprising the step of administration of at least one botulinum toxin serotype in a pharmaceutical composition having a low immunogenicity, thereby treating the domesticated animal without the animal developing immunity to the botulinum toxin.

20. The method of claim 19, wherein the pharmaceutical composition comprises a polysacharide that stabilizes the botulinum toxin.

21. The method of claim 19, wherein the polysacharide is a hydroxyethyl starch.

22. The method of claim 19, wherein the botulinum toxin treats pain of the domesticated animal.

23. The method of claim 19, wherein the domesticated animal is injured.

24. The method of claim 19, wherein the botulinum toxin is administered to facilitate recovery of the domesticated animal from surgery.

25. The method of claim 19, wherein the botulinum toxin is administered intramuscularly.

26. The method of claim 19, wherein the botulinum toxin is administered intrathecally.

27. The method of claim 19, wherein the botulinum toxin is botulinum toxin type A.

28. The method of claim 19, wherein the botulinum toxin is botulinum toxin type B.

29. The method of claim 19, comprising administration of a plurality of botulinum toxin serotypes selected from the group consisting of botulinum toxin serotypes A, B, C₁, D, E, F, and G.

30. The method of claim 19, wherein the botulinum toxin is a purified botulinum toxin.

31. The method of claim 19, wherein the botulinum toxin is a modified botulinum toxin.
